(19) United States
(12) Patent Application Publication
(10) Pub. No.: US 2026/0283527 A1
(43) Pub. Date: Sep. 24, 2026

(54) BIOLOGICAL SIGNAL DETECTION DEVICE AND BIOLOGICAL SIGNAL DETECTION SYSTEM

(71) Applicant: SONY GROUP CORPORATION, TOKYO (JP)

(72) Inventors: RYO SASAKI, TOKYO (JP); KAZUNARI YOSHIFUJI, TOKYO (JP); MAO KATSUHARA, TOKYO (JP); MASAHIRO SATO, TOKYO (JP)

(21) Appl. No.: 19/471,888

(22) PCT Filed: Apr. 17, 2024
(86) PCT No.: PCT/JP2024/015206

§ 371 Date: Oct. 2, 2025

(30) Foreign Application Priority Data
Apr. 28, 2023 (JP) 2023-075322

(51) Int. Cl. A61B 5/291

(52) U.S. Cl. CPC A61B 5/291

(57) ABSTRACT

A biological signal detection device according to an embodiment of the present disclosure includes a first measurer and a second measurer. The first measurer and the second measurer each include a first electrode and a second electrode that are configured to be in contact with a living body, and a first passive element that is electrically coupled between the second electrode and a reference potential line. The first measurer and the second measurer are each configured to output a first signal based on an electric potential of the first electrode.

### TECHNICAL FIELD

The present disclosure relates to a biological signal detection device and a biological signal detection system.

### BACKGROUND ART

A biological signal detection device has been proposed which includes a reference electrode disposed in a recess of an auricle and a detection electrode configured to be in contact with skin on a temporal bone, and generates a potential detection signal based on an electric potential of the reference electrode and an electric potential of the detection electrode (PTL 1).

### CITATION LIST

### Patent Literature

PTL 1: Japanese Unexamined Patent Application Publication No. 2018-186934

### SUMMARY OF THE INVENTION

In a device that detects a biological signal, it is desirable to improve detection performance.

It is desirable to provide a biological signal detection device having favorable detection performance.

A biological signal detection device according to an embodiment of the present disclosure includes a first measurer and a second measurer. The first measurer and the second measurer each include a first electrode and a second electrode that are configured to be in contact with a living body, and a first passive element that is electrically coupled between the second electrode and a reference potential line. The first measurer and the second measurer are each configured to output a first signal based on an electric potential of the first electrode.

A biological signal detection system according to an embodiment of the present disclosure includes a first measurer, a second measurer, and a signal processing device. The first measurer and the second measurer each include a first electrode and a second electrode that are configured to be in contact with a living body, and a first passive element that is electrically coupled between the second electrode and a reference potential line. The first measurer and the second measurer are each configured to output a first signal based on an electric potential of the first electrode. The signal processing device is configured to generate a second signal related to the living body, on a basis of the first signal to be generated by the first measurer and the first signal to be generated by the second measurer.

### BRIEF DESCRIPTION OF DRAWINGS

FIG. 1 is a diagram illustrating an example of a schematic configuration of a biological signal detection device according to an embodiment of the present disclosure.

FIG. 2 is a diagram for describing a configuration example of the biological signal detection device according to the embodiment of the present disclosure.

FIG. 3A is a diagram for describing a configuration example of the biological signal detection device according to the embodiment of the present disclosure.

FIG. 3B is a diagram for describing a configuration example of the biological signal detection device according to the embodiment of the present disclosure.

FIG. 3C is a diagram for describing a configuration example of the biological signal detection device according to the embodiment of the present disclosure.

FIG. 3D is a diagram for describing a configuration example of the biological signal detection device according to the embodiment of the present disclosure.

FIG. 3E is a diagram for describing a configuration example of the biological signal detection device according to the embodiment of the present disclosure.

FIG. 3F is a diagram for describing a configuration example of the biological signal detection device according to the embodiment of the present disclosure.

FIG. 4 is a diagram illustrating a configuration example of a measurer of a biological signal detection device according to Modification example 1 of the present disclosure.

FIG. 5 is a diagram illustrating another configuration example of the measurer of the biological signal detection device according to Modification example 1 of the present disclosure.

FIG. 6 is a diagram illustrating a configuration example of a measurer of a biological signal detection device according to Modification example 2 of the present disclosure.

FIG. 7 is a diagram illustrating another configuration example of the measurer of the biological signal detection device according to Modification example 2 of the present disclosure.

FIG. 8 is a diagram illustrating another configuration example of the measurer of the biological signal detection device according to Modification example 2 of the present disclosure.

FIG. 9 is a diagram illustrating a configuration example of a measurer of a biological signal detection device according to Modification example 3 of the present disclosure.

FIG. 10 is a diagram illustrating another configuration example of the measurer of the biological signal detection device according to Modification example 3 of the present disclosure.

FIG. 11 is a diagram illustrating another configuration example of the measurer of the biological signal detection device according to Modification example 3 of the present disclosure.

FIG. 12 is a diagram illustrating another configuration example of the measurer of the biological signal detection device according to Modification example 3 of the present disclosure.

FIG. 13 is a diagram illustrating another configuration example of the measurer of the biological signal detection device according to Modification example 3 of the present disclosure.

FIG. 14 is a diagram illustrating another configuration example of the measurer of the biological signal detection device according to Modification example 3 of the present disclosure.

FIG. 15 is a diagram illustrating a configuration example of a biological signal detection device according to Modification example 4 of the present disclosure.

FIG. 16 is a diagram illustrating a configuration example of a biological signal detection system according to Modification example 4 of the present disclosure.

FIG. 17 is a diagram illustrating a configuration example of the biological signal detection system according to Modification example 4 of the present disclosure.

FIG. 18 is a diagram illustrating a configuration example of the biological signal detection system according to Modification example 4 of the present disclosure.

### MODES FOR CARRYING OUT THE INVENTION

Some embodiments of the present disclosure are described below in detail with reference to the drawings. It is to be noted that description is given in the following order.
0. Background
1. Embodiment
2. Modification Examples
   2-1. Modification Example 1
   2-2. Modification Example 2
   2-3. Modification Example 3
   2-4. Modification Example 4

### 0. Background

A brainwave measurement device that measures, for example, a brainwave as biological information generally has a configuration in which a measurement electrode (a detection electrode), a reference electrode, and a signal processor are coupled to each other by wiring. The measurement electrode is disposed at a position near a brain activity site and detects a biological signal. The reference electrode detects the biological signal serving as a reference in a portion where propagation of the brainwave is small, such as an earlobe. In order to observe a brainwave having a large signal level to estimate a biological state with high accuracy, it is necessary to keep the reference electrode and the measurement electrode (the detection electrode) sufficiently away from each other, and to dispose the reference electrode at a limited position, which can constrain miniaturization of a brainwave meter. In recent years, in order to improve usability by miniaturization, efforts to acquire a brainwave in a limited area such as in an ear or around an ear have attracted attention.

### 1. Embodiment

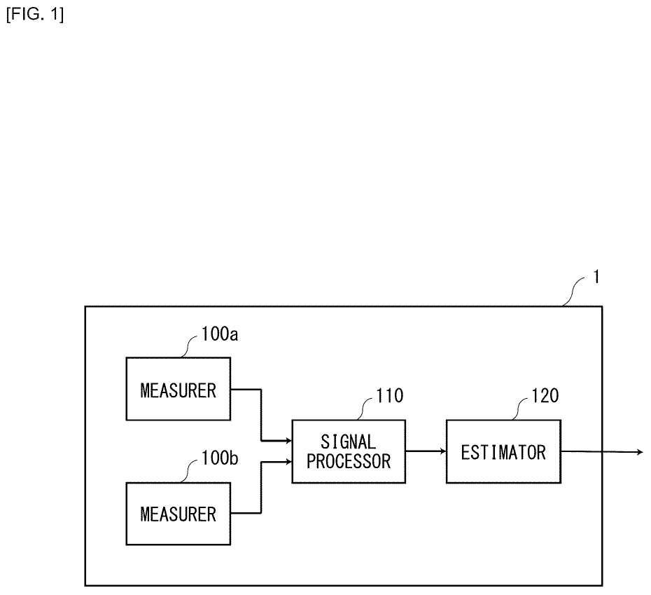

FIG. 1 is a diagram illustrating an example of a schematic configuration of a biological signal detection device according to an embodiment of the present disclosure. The biological signal detection device 1 is a device configured to detect a signal (hereinafter referred to as a biological signal) related to a living body. The biological signal is, for example, a signal related to an internal state of a human body serving as the living body. The biological signal detection device 1 includes a measurer 100 having a plurality of electrodes configured to be in contact with the living body, and is configured to generate the biological signal.

The biological signal is, for example, a signal related to an electric potential (a voltage) generated with activity of the living body. Specific examples thereof include a brainwave that is a signal associated with cerebral activity, a cardiac signal associated with cardiac activity, and a myoelectric signal associated with muscular activity, and the like. The biological signal is a signal related to, for example, the brainwave, a cardiac potential, a myoelectric potential, a pulse wave, or the like.

The biological signal detection device 1 detects the biological signal such as the signal related to the brainwave (a brainwave signal). The brainwave signal to be detected is, for example, an electric signal corresponding to an activity state of a brain of a user. The biological signal detection device 1 acquires the biological signal, thereby being configured to check a state of the living body. The biological signal detection device 1 is a device configured acquire the biological signal, and may also be referred to as a biological signal acquiring device.

The biological signal detection device 1 is applicable to, for example, an electronic apparatus worn by the user. The biological signal detection device 1 may be applied to the electronic apparatus that is wearable on a body including an ear, a head, a face, a neck, a hand, a wrist, an arm, a foot, a chest, etc. The biological signal detection device 1 may be implemented as a device to be mounted on wearable devices including, for example, earphones, headphones, and eyewear.

The biological signal detection device 1 includes a plurality of measurers 100 (a measurer 100a and a measurer 100b in FIG. 1) and a signal processor 110. The biological signal detection device 1 (or the measurer 100) may be configured, for example, as a sensor (a biological sensor) that is configured to acquire the biological signal. The biological signal detection device 1 may include an estimator 120, as in the example illustrated in FIG. 1. It is to be noted that the estimator 120 may be provided in the signal processor 110 or may be provided separately from the signal processor 110.

The measurer 100 (each of the measurer 100a and the measurer 100b) is a measurement circuit and is configured to generate the biological signal. The measurer 100 includes a plurality of electrodes for detecting an electric potential, which will be described later. The measurer 100 is, for example, configured to execute brainwave measurement and configured to detect a brainwave signal as the biological signal. The measurer 100 may acquire the biological signal of the user and output the brainwave signal to the signal processor 110. The measurer 100 may be referred to as a measurement unit configured to acquire the biological signal.

The signal processor 110 is a signal processing circuit and is configured to execute a signal process (an information process). The signal processor 110 includes, for example, a processor and memories (ROM, RAM and the like), and is configured to perform various signal processes. The signal processor 110 may read and execute a program incorporated therein, and may perform the signal process (the information process).

The signal processor 110 is configured to execute the signal process on a signal to be inputted. The signal processor 110 includes, for example, a circuit that performs various signal processes on the biological signal. The signal processor 110 may perform various signal processes including, for example, a process of reducing noises, a frequency-analysis process, and a normalization process on the biological signal to be outputted from the plurality of measurers 100.

The estimator 120 is configured to execute a process of estimating the state of the living body with use of the biological signal. The estimator 120 may include a processor, a memory, and the like. The estimator 120 performs, for example, a process of calculating a feature quantity using the biological signal after being subjected to a noise-reduction process. The estimator 120 extracts the feature quantity by performing, for example, a Fast Fourier Transform (FFT) process or a wavelet analysis process on the biological signal. The feature quantity is, for example, a component of an α wave, a component of a β wave, a component of a y wave, or the like included in the biological signal.

The estimator 120 estimates the state of the living body on the basis of a result of the calculation of the feature quantity. The estimator 120 estimates the state of, for example, whether or not the living body is relaxed, whether or not the living body is concentrating, and the like, on the basis of the component of the α wave and the component of the β wave. The estimator 120 may be referred to as a determiner configured to determine the state of the living body.

As another example, the estimator 120 may determine whether or not the living body is in a sleeping state with use of the biological signal related to the brainwave. Further, for example, the estimator 120 may estimate a heart rate with use of the biological signal related to the cardiac potential. The estimator 120 may estimate an emotion of the living body by analyzing the biological signal. Analyzing the biological signal as described above makes it possible to check the state of the living body.

The estimator 120 is configured to generate state information that is information indicating the state of the living body. The estimator 120 may generate and output the state information as an estimation result. The state information includes, for example, information indicating whether or not the living body is in a relaxed state, information indicating whether or not the living body is in a sleeping state, information indicating a psychological state such as an emotion of the living body, and information indicating a heart rate.

The state information may be used, for example, for displaying an image indicating the state of the living body, outputting a sound indicating the state of the living body, and the like. The biological signal detection device 1 may include a display unit configured to display an image based on the state information, for example, an organic EL display, a liquid crystal display, or the like. The display unit may include a touch panel.

The biological signal detection device 1 may include a sound output unit, such as a speaker, that is configured to output a sound (a music piece(BGM), a sound effect, or the like) based on the state information. The biological signal detection device 1 may be configured to apply a vibration based on the state information to the user. The biological signal detection device 1 may include a presentation unit (a display unit, a sound output unit, or the like) configured to present (provide) an image, a sound, a vibration, or the like to the user.

It is to be noted that the signal processor 110, the estimator 120, or both may be provided in an external device disposed outside of the biological signal detection device 1. Examples of the external device include an electronic apparatus that is a terminal device (a terminal) to be used by the user, a server (for example, a cloud server), and the like. Examples of the electronic apparatus include a smartphone, a tablet terminal, a wearable terminal, a computer, and another information processing device.

A combination of the biological signal detection device 1 and the external device may also be referred to as a biological signal detection device. The biological signal detection device 1 and the external device may transmit and receive a signal (information) via radio or wired communication. For example, the biological signal detection device 1 is configured to be coupled to a network and to communicate with the external device. A combination of the biological signal detection device 1 and the external device coupled to each other via the network may also be referred to as a biological signal detection device or a biological signal detection system.

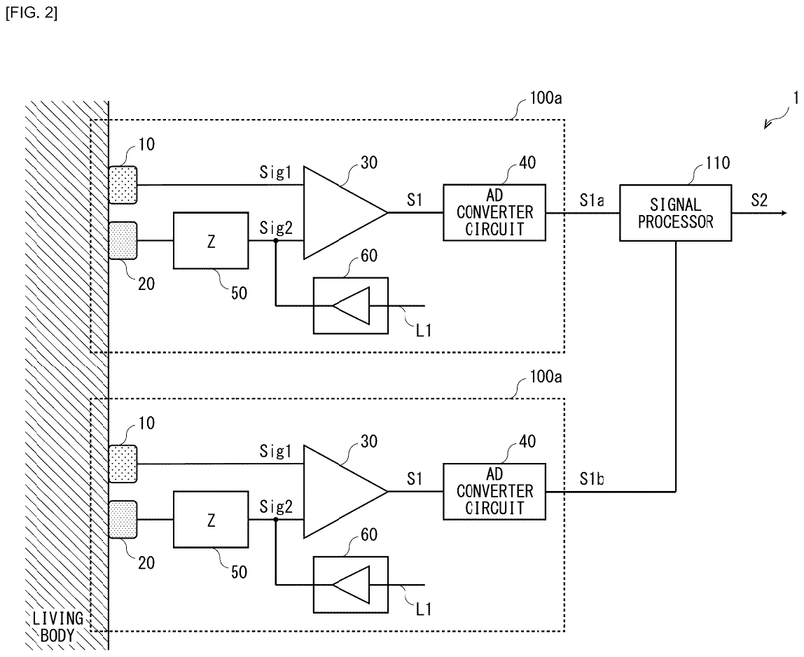

FIG. 2 is a diagram for describing a configuration example of the biological signal detection device according to the embodiment of the present disclosure. The biological signal detection device 1 includes two measurers 100 (the measurer 100a and the measurer 100b). The measurer 100a and the measurer 100b have the same configuration as each other. In a case where the measurer 100a and the measurer 100b are not distinguished from each other in particular, the measurer 100a and the measurer 100b are also simply referred to as the measurer 100.

The measurer 100 includes a plurality of electrodes to be used for detecting an electric potential, and is configured to measure the electric potential of the living body. As in the example illustrated in FIG. 2, the measurer 100 includes a plurality of electrodes (a measurement electrode 10 and a bias electrode 20 in FIG. 2), an amplifier circuit 30, and an AD converter circuit 40.

The measurement electrode 10 and the bias electrode 20 are each configured to be in contact with the living body. The measurement electrode 10 and the bias electrode 20 are provided away from each other, and are in contact with respective positions that are different from each other. The measurement electrode 10 is configured to be disposed at any position within an area in which the biological signal is to be acquired. The bias electrode 20 is configured to be disposed at any position including the vicinity of the measurement electrode 10.

The measurer 100 detects an electric potential (a voltage) of a surface of the living body with use of the measurement electrode 10 and the bias electrode 20. For example, an electric potential difference may occur between the measurement electrode 10 and the bias electrode 20 that are in contact with the skin of the living body due to electricity generated inside the living body.

The measurement electrode 10 and the bias electrode 20 may be disposed, for example, with a gap (a distance) of less than or equal to a range from 30 mm to 40 mm both inclusive, which is regarded as space resolution of the brainwave. The gap between the measurement electrode 10 and the bias electrode 20 may be, for example, less than or equal to 30 mm. Further, for example, the gap between the measurement electrode 10 and the bias electrode 20 may be less than or equal to 20 mm.

The measurement electrode 10 and the bias electrode 20 each include an electrically conductive material. The measurement electrode 10 and the bias electrode 20 each include, for example, aluminum (Al), copper (Cu), silver/silver chloride (Ag/AgCl), gold (Au), or the like. It is to be noted that each of the measurement electrode 10 and the bias electrode 20 include another electrically conductive material.

The measurement electrode 10 and the bias electrode 20 may each include another material such as an electrically conductive and elastic resin material. A shape of each of the measurement electrode 10 and the bias electrode 20 is not particularly limited, and may be a circular shape, an oval shape, or another shape. In addition, the numbers and arrangements of the measurement electrode 10 and the bias electrode 20 are not limited to the illustrated example.

The measurer 100 of the biological signal detection device 1 includes a connection circuit 50 and a supplier 60 as in the example illustrated in FIG. 2. The connection circuit 50 is provided between the bias electrode 20 and the supplier 60, and electrically couples the bias electrode 20 and the supplier 60 to each other.

The connection circuit 50 includes a passive element and has a predetermined impedance Z. The connection circuit 50 includes, for example, a plurality of passive elements including a resistor, a capacitor, and the like. In the example illustrated in FIG. 2, one end of the connection circuit 50 is electrically coupled to the bias electrode 20. Another end of the connection circuit 50 is electrically coupled to the supplier 60 and the amplifier circuit 30.

The impedance Z of the connection circuit 50 is set, for example, to a value greater than an impedance (a contact impedance) between the living body and the bias electrode 20. The impedance Z of the connection circuit 50 may be 10 times or more of the impedance between the living body and the bias electrode 20. The impedance Z, e.g., a resistance value, of the connection circuit 50 may be set to a value greater than or equal to 1 MΩ.

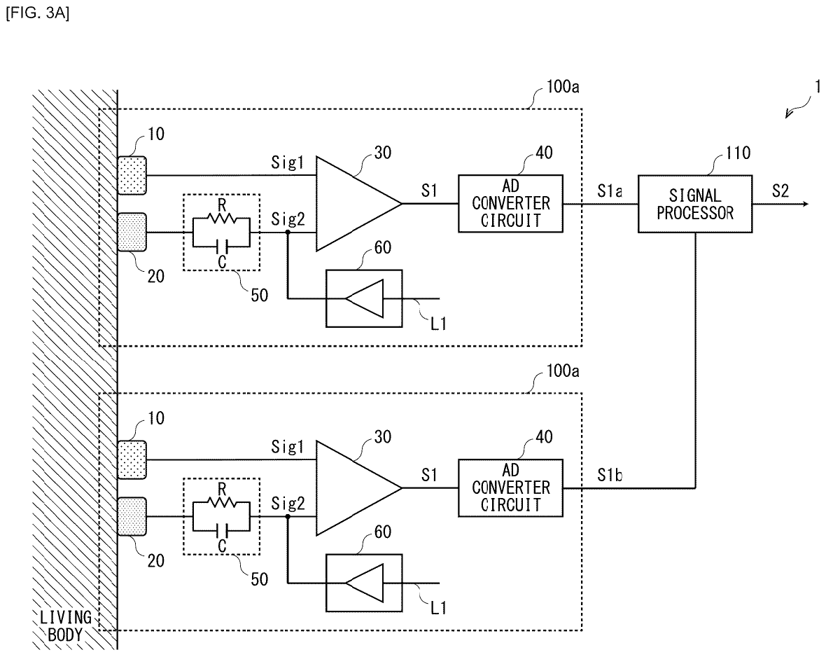

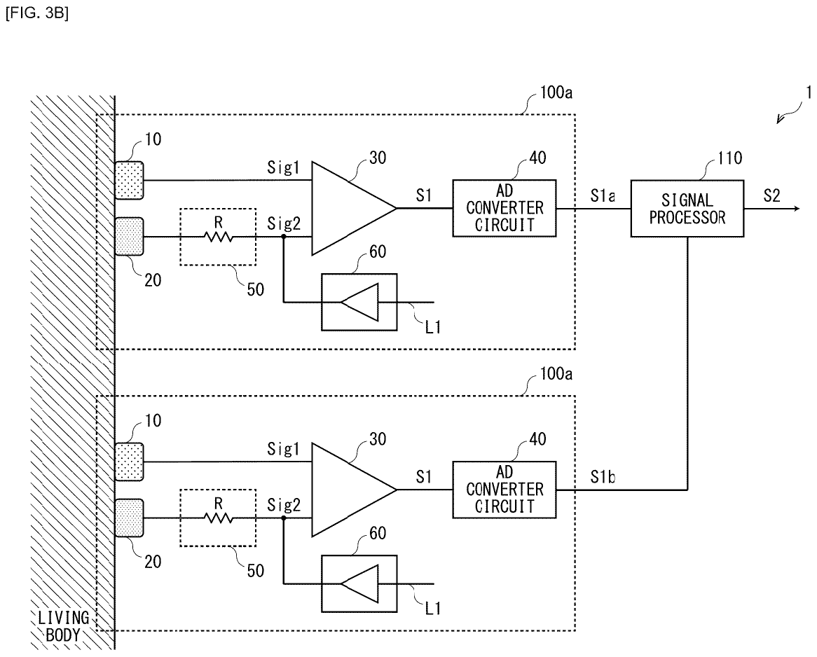

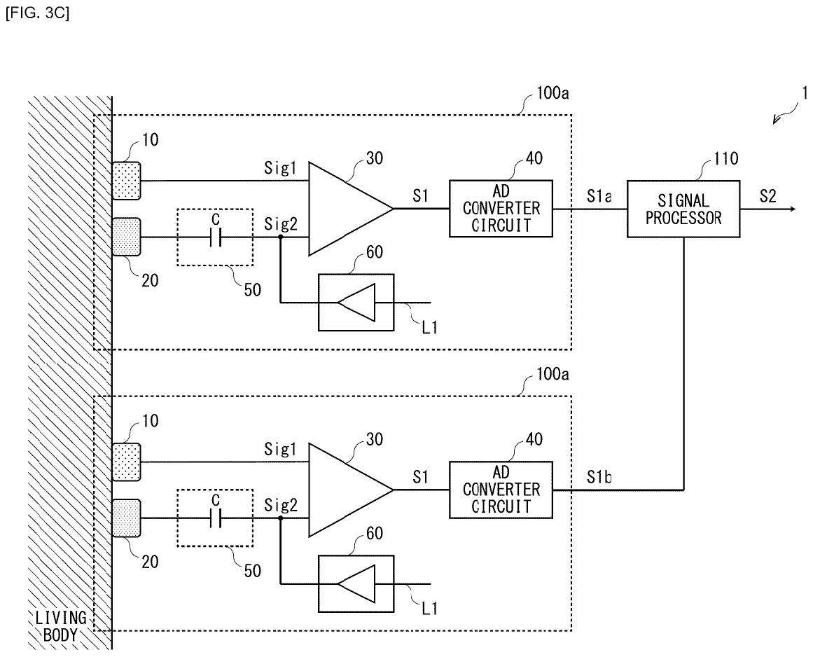

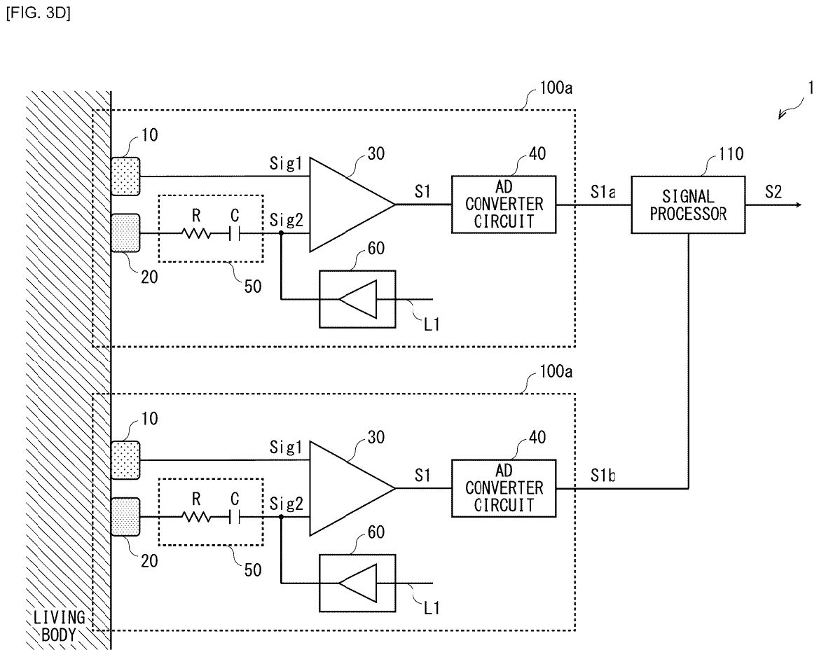

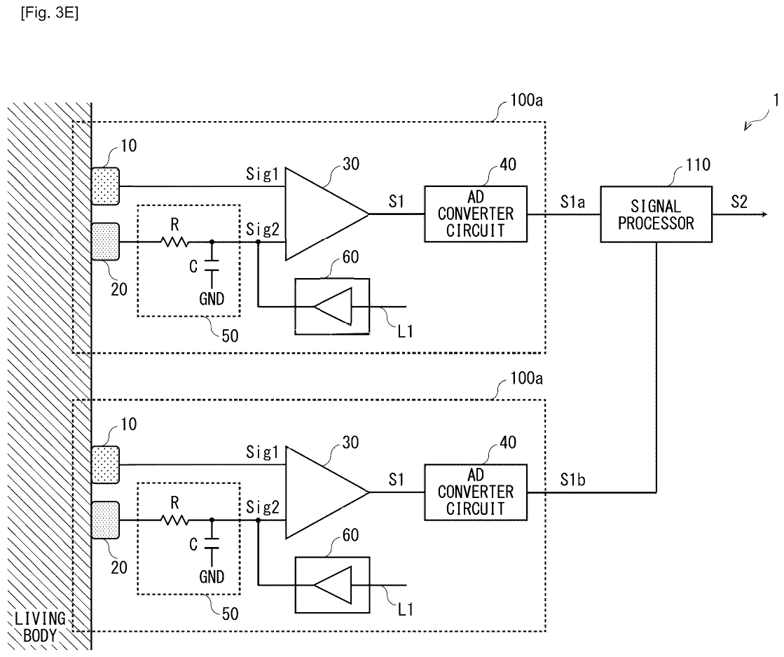

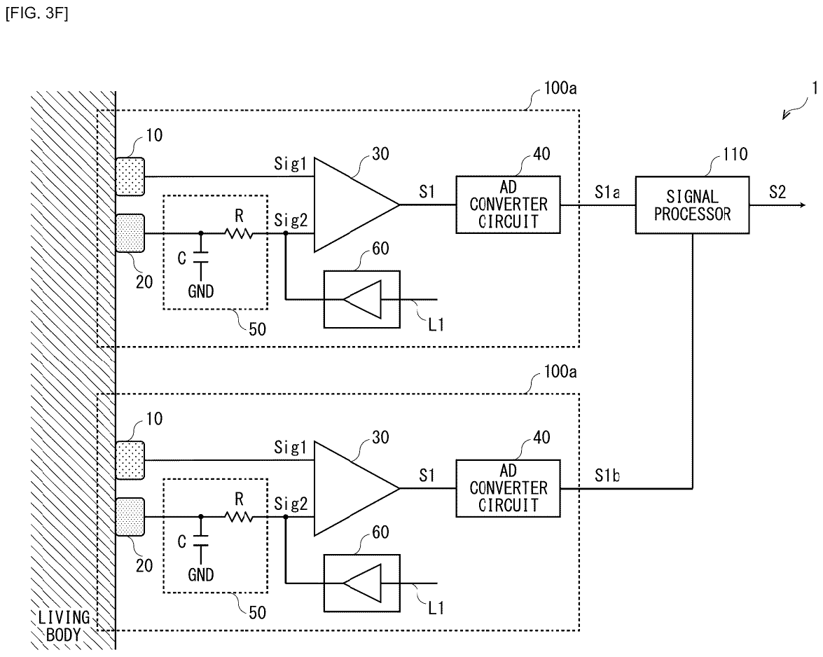

As illustrated in FIG. 3A, the connection circuit 50 may include, for example, a resistor R and a capacitor C that are coupled to each other in parallel. The resistor R is electrically coupled between a reference potential line L1 and the bias electrode 20. The capacitor C is electrically coupled between the reference potential line L1 and the bias electrode 20. A resistance value of the resistor R may be greater than or equal to 1 MΩ. Further, the resistance value of the resistor R may be greater than or equal to 5 MΩ, or may be greater than or equal to 10 MΩ.

It is to be noted that a configuration example of the connection circuit 50 is not limited to the example illustrated in FIG. 3A, and may be changed as appropriate. The connection circuit 50 may have respective configurations illustrated in FIGS. 3B to 3F. The connection circuit 50 may include a resistor and a capacitor that are coupled to each other in series. The connection circuit 50 may have a plurality of resistors and a plurality of capacitors. Further, the connection circuit 50 may include an inductor.

The supplier 60 is a supplier a (supplier circuit) configured to supply a voltage, and is configured to output the voltage to the connection circuit 50 and the bias electrode 20. As in the example schematically illustrated in FIG. 2, for example, the supplier 60 includes a buffer circuit electrically coupled to the reference potential line L1.

The supplier 60 supplies, for example, a reference potential or a GND (ground) potential to be supplied via the reference potential line L1 to connection circuit 50. For example, the supplier 60 outputs a reference potential VREF to be provided by the reference potential line L1 to the connection circuit 50 by the buffer circuit.

The bias electrode 20 and the reference potential line L1 are electrically coupled to each other via the connection circuit 50 and the supplier 60. This makes it possible to set a relative electric potential between the measurer 100 and the living body. The bias electrode 20 serves as an electrode for determining the relative electric potential between the measurer 100 and the living body. The bias electrode 20 may also be referred to as an electrode for the reference potential. The electric potential of the bias electrode 20 serves as the reference potential with respect to a measurement signal.

The measurement electrode 10 comes into contact with a measurement site (a point to be measured) in actual use, and an electric potential of the contact site is given. The measurement electrode 10 is disposed, for example, directly on an activity region, of the living body, in which the biological signal is to be measured. The measurement electrode 10 is electrically coupled to the amplifier circuit 30 and supplies a measurement signal Sig1 to the amplifier circuit 30. The measurement signal Sig1 is a signal corresponding to the electric potential of the contact site of the living body. The measurement signal Sig1 is a biological signal obtained by the measurement electrode 10.

The bias electrode 20 comes into contact with the living body at a position that differs from the position of the measurement electrode 10 in actual use, and an electric potential of the contact site is given. The bias electrode 20 may be disposed at any position around the measurement electrode 10, for example. The bias electrode 20 is electrically coupled to the amplifier circuit 30 via the connection circuit 50, for example, and supplies a measurement signal Sig2 to the amplifier circuit 30. The measurement signal Sig2 corresponds to the electric potential of the contact site of the living body. The measurement signal Sig2 is a biological signal obtained by the bias electrode 20.

The amplifier circuit 30 is configured to amplify a signal to be inputted. The amplifier circuit 30 includes, for example, a differential amplifier circuit (a differential amplifier) configured to amplify a signal. The amplifier circuit 30 (a differential amplifying unit) generates a biological signal S1 on the basis of, for example, a measurement signal obtained by the measurement electrode 10 and a measurement signal obtained by the bias electrode 20. The amplifier circuit 30 may also be referred to as a signal detector (or a signal generator) configured to detect a biological signal.

In the example illustrated in FIG. 2, the measurement signal Sig1 is inputted by the measurement electrode 10 to the amplifier circuit 30, and the measurement signal Sig2 is inputted by the bias electrode 20 to the amplifier circuit 30. The amplifier circuit 30 generates the biological signal S1 based on a difference between the measurement signal Sig1 and the measurement signal Sig2. The amplifier circuit 30 may generate the biological signal S1 corresponding to the difference between an electric potential of the measurement signal Sig1 and an electric potential of the measurement signal Sig2, and may output the generated biological signal S1 to the AD converter circuit 40.

The AD converter circuit 40 is configured to convert an analog signal to be inputted into a digital signal. The AD converter circuit 40 is an ADC (Analog to Digital Converter). The biological signal S1 is inputted to the AD converter circuit 40 from the amplifier circuit 30. The AD converter circuit 40 performs an AD conversion process on the biological signal S1.

The AD converter circuit 40 (an AD converter) converts the biological signal S1 that is the analog signal outputted by the amplifier circuit 30 into the digital signal. The AD converter circuit 40 may output the biological signal S1 converted into the digital signal to the signal processor 110. The signal processor 110 may acquire the biological signals S1 from the respective measurers 100 (in FIG. 2, a biological signal S1a detected by the measurer 100a and a biological signal S1b detected by the measurer 100b), and may perform a noise reduction process, a frequency analysis process, and the like on the biological signals S1.

In a case where the biological signal is to be detected, a noise propagating throughout the body (e.g., a noise component of a particular frequency, for example, 50 Hz) can be mixed into the biological signal as a common mode noise, and a signal quality of the biological signal can be degraded. Accordingly, in a case where the common mode noise, which is a common noise component, is large, it can make it difficult to accurately estimate the state of the living body using the biological signal. In particular, in a case where the distance between the electrodes for measurement is short, the quality of the biological signal tends to be degraded.

Accordingly, the biological signal detection device 1 according to the present embodiment may detect the biological signal including the common mode noise by the measurer 100a and the measurer 100b. The measurer 100a and the measurer 100b each include the measurement electrode 10, and the passive element that is electrically coupled between the bias electrode 20 and the reference potential line L1. The signal processor 110 is configured to obtain the biological signal with reduced common mode noise by performing calculation using the biological signal Sla and the biological signal S1b respectively outputted from the measurer 100a and the measurer 100b.

The signal processor 110 is configured to generate a biological signal S2 on the basis of the biological signal Sla obtained by the measurer 100a and the biological signal S1b obtained by the measurer 100b. The signal processor 110 is configured to generate the biological signal S2 based on a difference between the biological signal S1a detected by the measurer 100a and the biological signal S1b detected by the measurer 100b, for example.

The signal processor 110 may generate the biological signal S2 with reduced common mode noise, for example, by performing a subtraction process between the biological signal S1a detected by the measurer 100a and the biological signal S1b detected by the measurer 100b. The subtraction process makes it possible to improve an S/N ratio and to obtain the biological signal with less noise.

As described above, the biological signal detection device 1 according to the present embodiment makes it possible to reduce the common mode noise included in the biological signal. It is possible to reduce the common mode noise included in the biological signal, and to improve the quality of the biological signal. This makes it possible to estimate the state of the living body with high accuracy.

In the present embodiment, the biological signal based on an electric potential of the measurement electrode 10 is detected, on the basis of an electric potential of the bias electrode 20 that is electrically coupled to the passive element (for example, the resistor R, the capacitor C, and the like described above) of the connection circuit 50. Accordingly, for example, it is possible to increase the difference between the measurement signal Sig1 and the measurement signal Sig2. Further, it is possible to detect the biological signal including the common mode noise, and to acquire the biological signal from which the common mode noise is removed by the subtraction between the biological signals.

In addition, in the present embodiment, even in the case where the contact position of the measurement electrode 10 and the contact position of the bias electrode 20 are close to each other, it is possible to perform the measurement of the biological signal S2 with high accuracy. This makes it possible to reduce a size of the biological signal detection device 1 (or the measurer 100), and to reduce wearing load on the user.

Further, the estimator 120 of the biological signal detection device 1 makes it possible to estimate the state of the living body with high accuracy, by using the biological signal S2 to be detected by the signal processor 110. It is possible to achieve the biological signal detection device 1 having high detection performance.

### [Workings and Effects]

The biological signal detection device 1 according to the embodiment includes a first measurer (the measurer 100a) and a second measurer (the measurer 100b). The first measurer and the second measurer each include a first electrode (the measurement electrode 10) and a second electrode (the bias electrode 20) that are configured to be in contact with the living body, and a first passive element (for example, the resistor R or the capacitor C) that is electrically coupled between the second electrode and a reference potential line (the reference potential line L1). The first measurer and the second measurer are each configured to output a first signal (the biological signal S1) based on an electric potential of the first electrode.

The biological signal detection device 1 according to the present embodiment includes the measurer 100a and the measurer 100b. The measurer 100a and the measurer 100b each include the measurement electrode 10, and the passive element (the resistor R, the capacitor C or the like) that is electrically coupled between the bias electrode 20 and the reference potential line L1. Accordingly, the measurer 100a and the measurer 100b detect the respective biological signals each including the common mode noise, and perform calculation between the biological signals, thereby making it possible to reduce the common mode noise. It is possible to achieve the biological signal detection device having favorable detection performance.

Next, description is given of modification examples of the present disclosure. Hereinafter, components similar to those in the foregoing embodiment are denoted by the same reference numerals, and descriptions thereof are omitted as appropriate.

### 2. Modification Examples

### (2-1. Modification Example 1)

Although a configuration example of the measurer 100 have been described in the above-described embodiment, the configuration is merely an example, and the configuration of the measurer 100 is not limited to the above-described example. In the above-described embodiment, the measurer 100 includes one bias electrode, but the number and arrangement of the bias electrodes are not limited thereto.

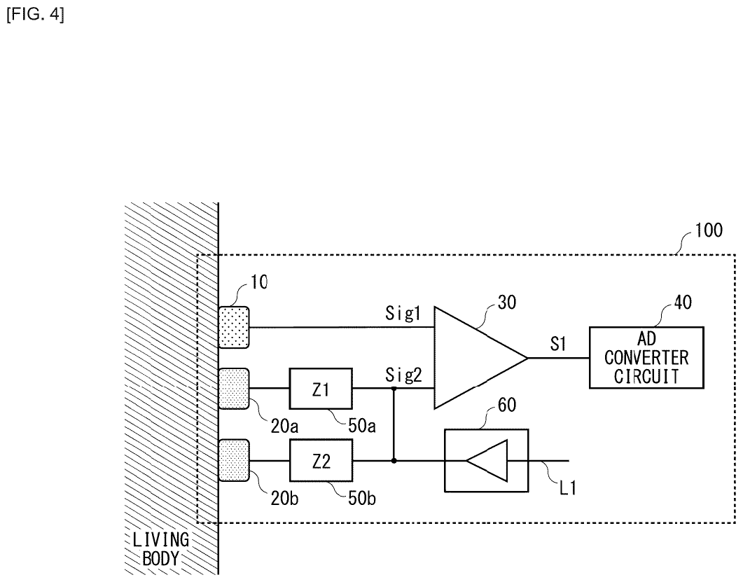

FIG. 4 is a diagram illustrating a configuration example of a measurer of a biological signal detection device according to Modification example 1 of the present disclosure. For example, the plurality of measurers 100 of the biological signal detection device 1 may each include a bias electrode 20a, a bias electrode 20b, a connection circuit 50a, and a connection circuit 50b, as illustrated in FIG. 4.

As illustrated in FIG. 4, the connection circuit 50a is electrically coupled between the bias electrode 20a and the reference potential line L1. The connection circuit 50b is electrically coupled between the bias electrode 20b and the reference potential line L1. Further, in the example illustrated in FIG. 4, the connection circuit 50a and the connection circuit 50b are both electrically coupled to the amplifier circuit 30.

The connection circuit 50a and the connection circuit 50b each include a passive element and has a predetermined impedance Z. The connection circuit 50a and the connection circuit 50b each include, for example, a plurality of passive elements including a resistor, a capacitor, and the like. The connection circuits 50a and 50b may each include an inductor.

In the embodiment illustrated in FIG. 4, the measurement signal Sig1 is inputted by the measurement electrode 10 to the amplifier circuit 30, and the measurement signal Sig2 is inputted by the bias electrodes 20a and 20b to the amplifier circuit 30. The amplifier circuit 30 may generate and output the biological signal S1 on the basis of the measurement signal Sig1 based on the electric potential of the measurement electrode 10 and the measurement signal Sig2 based on the electric potential of the bias electrode 20a and the bias electrode 20b.

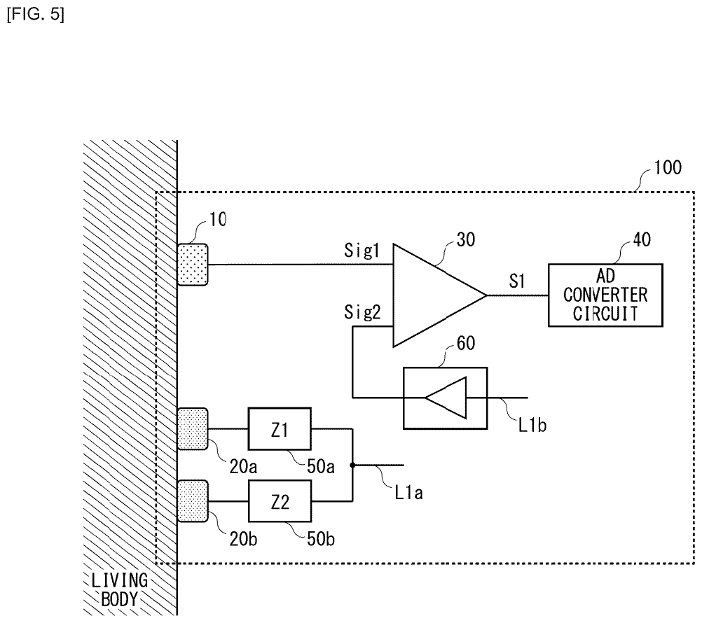

FIG. 5 is a diagram illustrating another configuration example of the measurer of the biological signal detection device according to Modification example 1. As in the example illustrated in FIG. 5, the bias electrode 20a and the bias electrode 20b may be supplied with the reference potential, e.g., a GND potential, by a reference potential line L1a.

Further, a signal having a specific electric potential (e.g., the reference potential VREF) or the GND potential may be inputted as the signal Sig2 from a reference potential line L1b to the amplifier circuit 30 via the supplier 60.

The biological signal detection device according to the present modification example also makes it possible to reduce the common mode noise, and to suppress degradation of the quality of the biological signal. It is possible to achieve the biological signal detection device having favorable detection performance.

### (2-2. Modification Example 2)

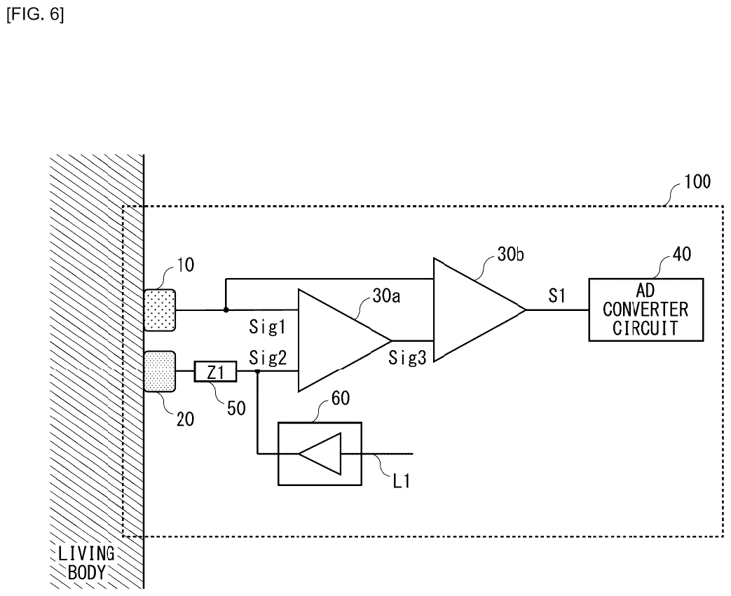

FIG. 6 is a diagram illustrating a configuration example of a measurer of a biological signal detection device according to Modification example 2. As in the example illustrated in FIG. 6, the measurer 100 may include an amplifier circuit 30a and an amplifier circuit 30b. The amplifier circuit 30a and the amplifier circuit 30b each include, for example, a differential amplifier circuit (a differential amplifier) configured to amplify a signal.

The amplifier circuit 30a is configured to generate a measurement signal Sig3 based on the measurement signal Sig1 and the measurement signal Sig2, and to output the measurement signal Sig3 to the amplifier circuit 30b. The amplifier circuit 30b is configured to generate the biological signal S1 on the basis of the measurement signal Sig1 and the measurement signal Sig3, and to output the biological signal S1 to the AD converter circuit 40.

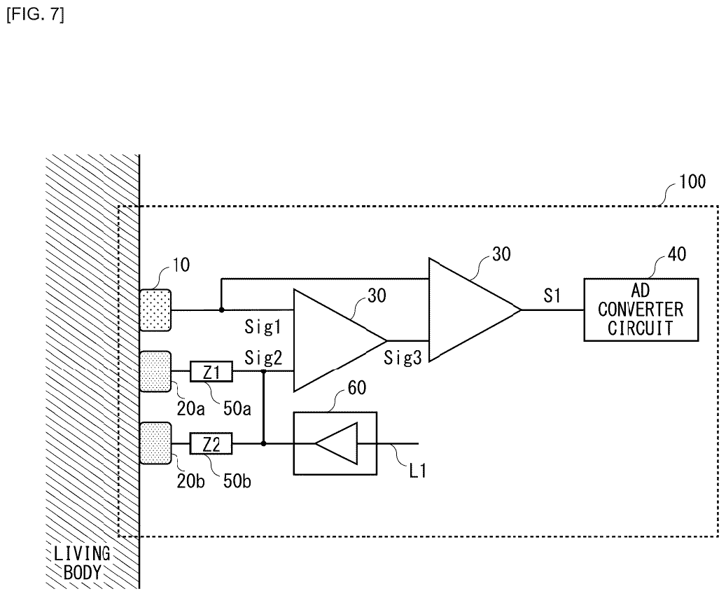

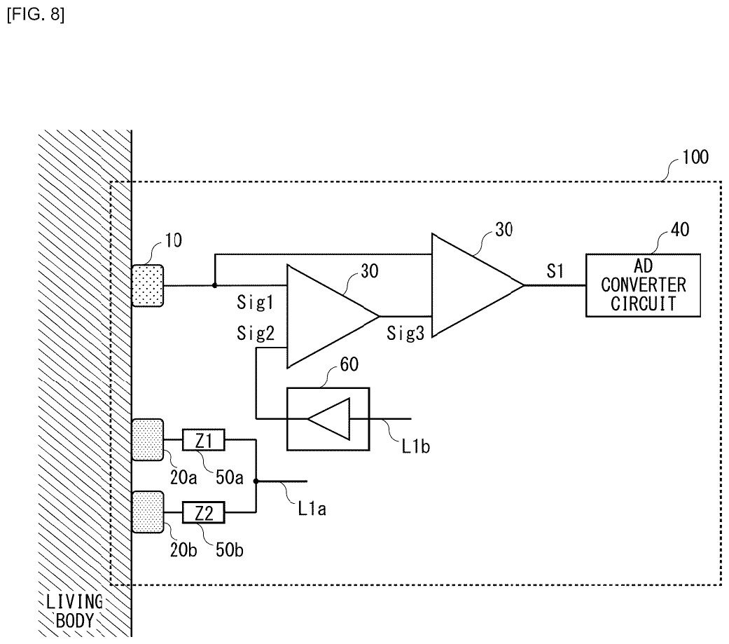

FIGS. 7 and 8 are each a diagram illustrating another configuration example of the measurer of the biological signal detection device according to Modification example 2. The measurer 100 may include the bias electrode 20a, the bias electrode 20b, the connection circuit 50a, and the connection circuit 50b, as illustrated in FIG. 7 or FIG. 8. In this case of the present modification example also, effects similar to those of the biological signal detection device according to the above-described embodiment are obtainable.

### (2-3. Modification Example 3)

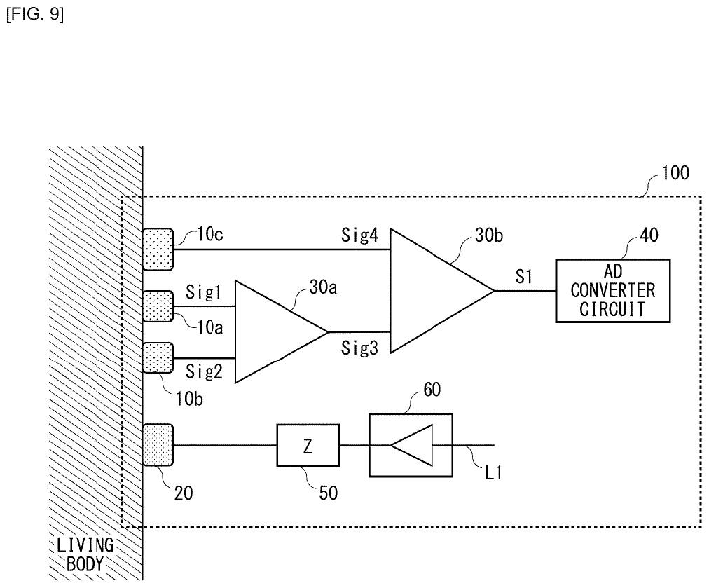

FIG. 9 is a diagram illustrating a configuration example of a measurer of a biological signal detection device according to Modification example 3. In the above-described embodiment, the example in which the measurer 100 includes one measurement electrode has been described, but the number and arrangement of the measurement electrodes are not limited thereto. The measurer 100 may include two or more measurement electrodes.

For example, each of the measurers 100 of the biological signal detection device 1 may include a measurement electrode 10a, a measurement electrode 10b, and a measurement electrode 10c, as in the example illustrated in FIG. 9. It is to be noted that the measurer 100 may include four or more measurement electrodes 10.

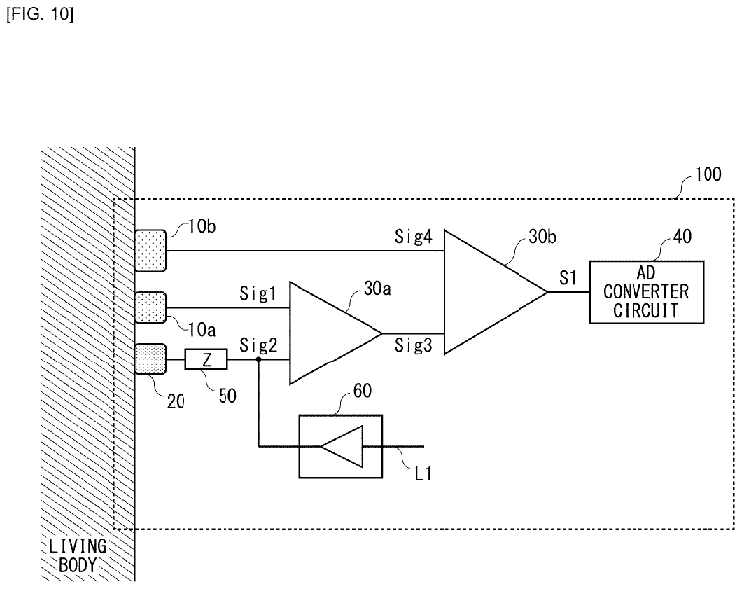

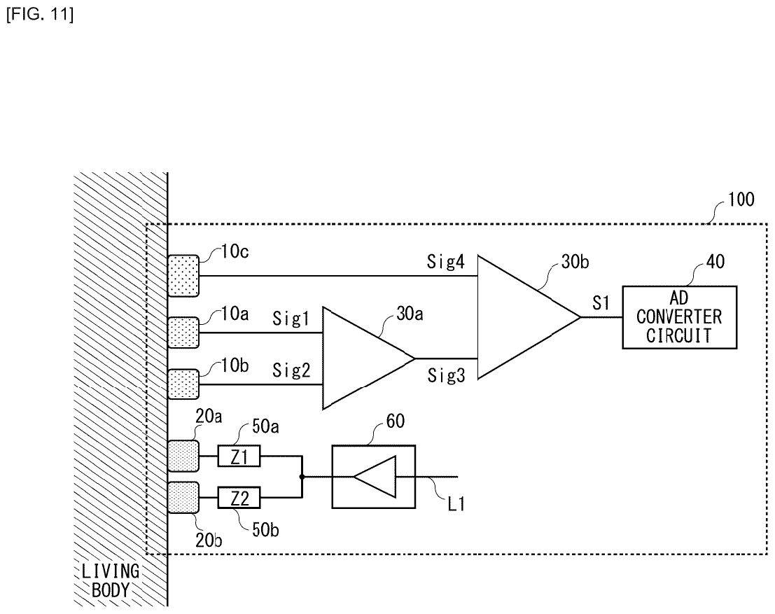

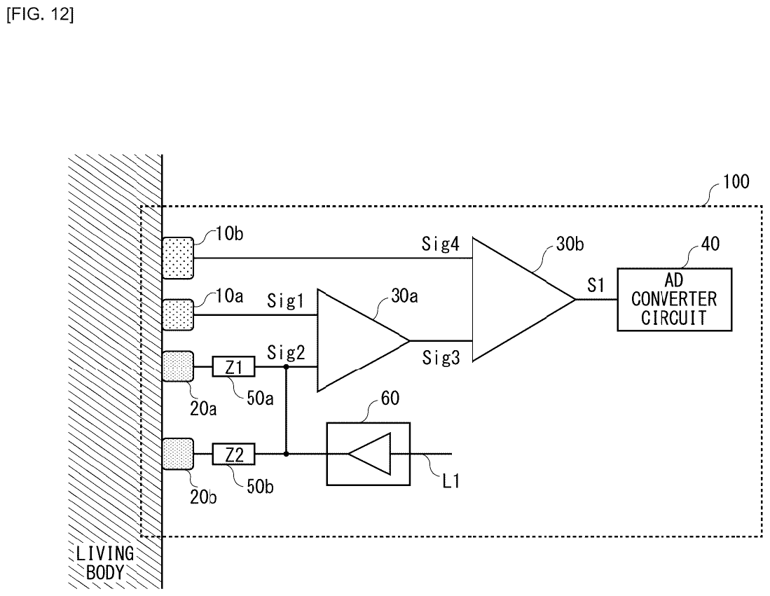

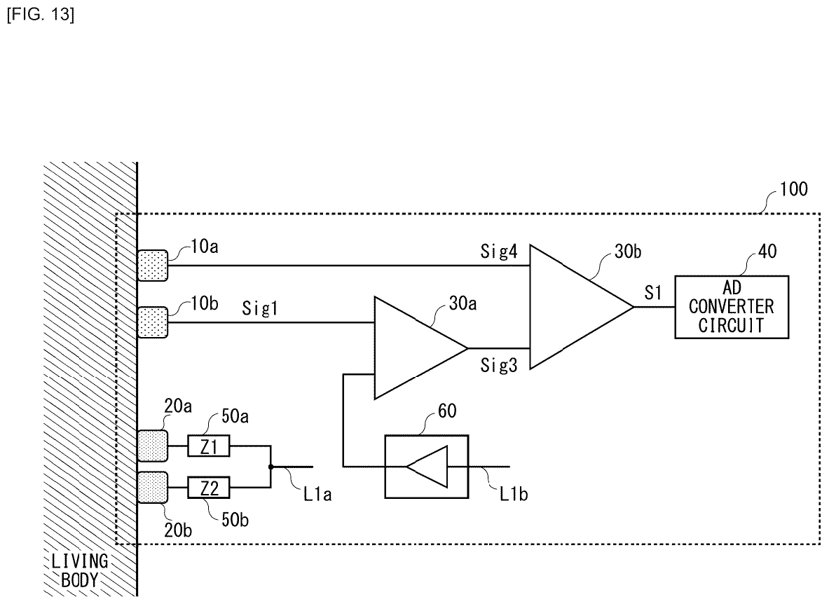

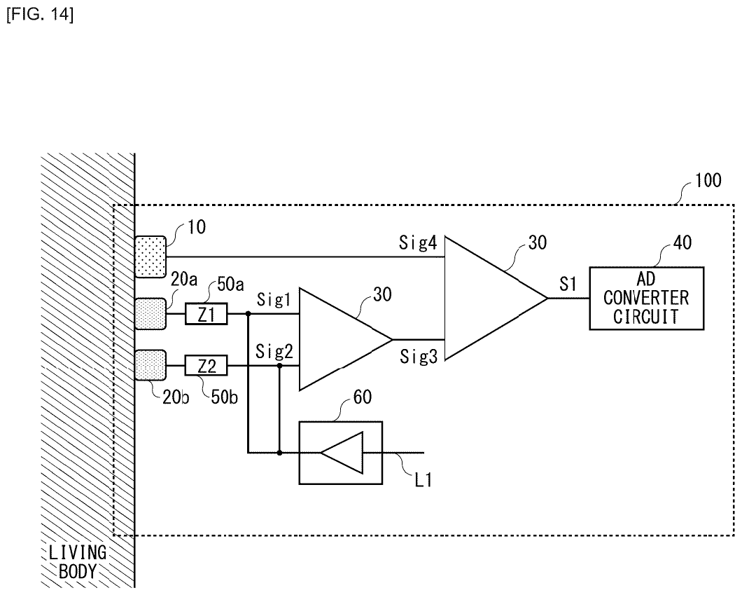

FIGS. 10 to 14 are each a diagram illustrating another configuration example of the measurer of the biological signal detection device according to Modification example 3. For example, the measurer 100 may have respective circuit configurations illustrated in FIGS. 10 to 14. As in the examples illustrated in FIGS. 10 to 14, the measurer 100 may have the measurement electrode 10a, the measurement electrode 10b, the measurement electrode 10c, the bias electrode 20a, the bias electrode 20b, etc., arranged therein. In this case of the present modification example also, effects similar to those of the biological signal detection device according to the above-described embodiment are obtainable.

### (2-4. Modification Example 4)

The technology according to the present disclosure is applicable to a variety of products. The biological signal detection device 1 according to the present disclosure may be applied to a wearable device such as an earphone device or a headphone device.

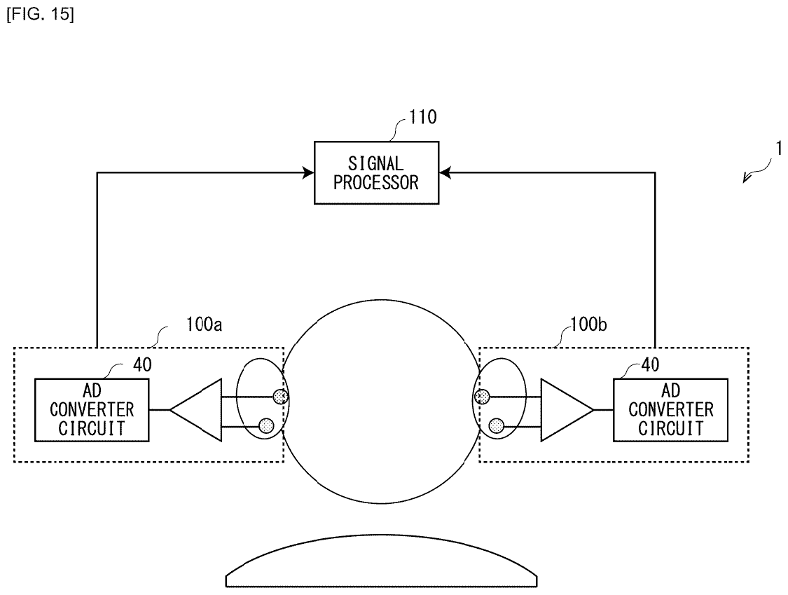

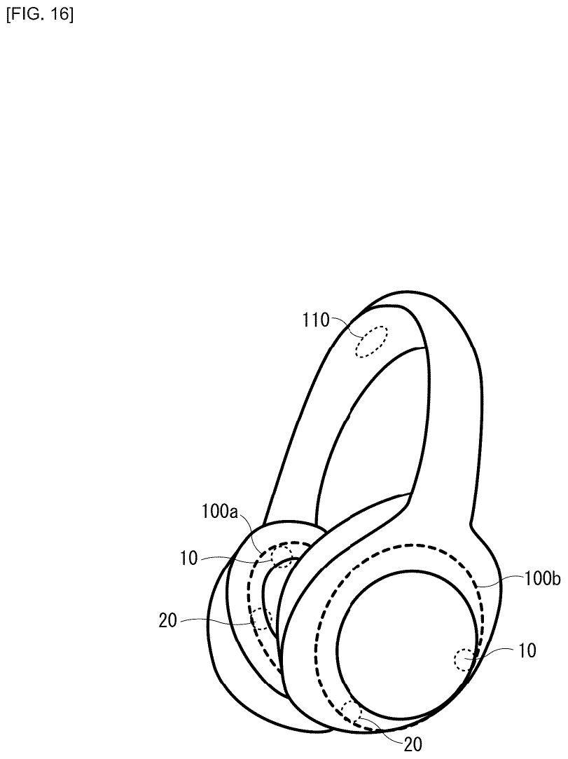

For example, as in the examples illustrated in FIG. 15 or FIG. 16, the biological signal detection device 1 according to the present disclosure may be applied to headphones. For example, the measurers 100a and 100b and the signal processor 110 may be mounted on each speaker unit of the headphones. For example, as in the example schematically illustrated in FIG. 15 or FIG. 16, the measurer 100a may be disposed with respect to the left ear of the user, and the measurer 100b may be disposed with respect to the right ear of the user.

It is to be noted that the signal processor 110 may be provided in an electronic apparatus 200 outside the biological signal detection device 1. The electronic apparatus 200 is, for example, a terminal device to be used by the user. The electronic apparatus 200 may include, for example, a smartphone, a tablet terminal, a wearable terminal, a computer, or another information processing device.

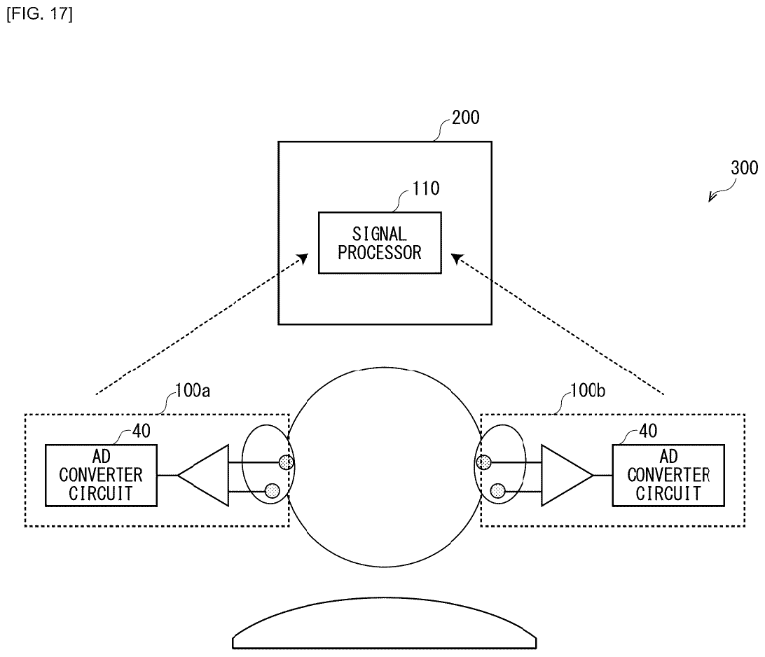

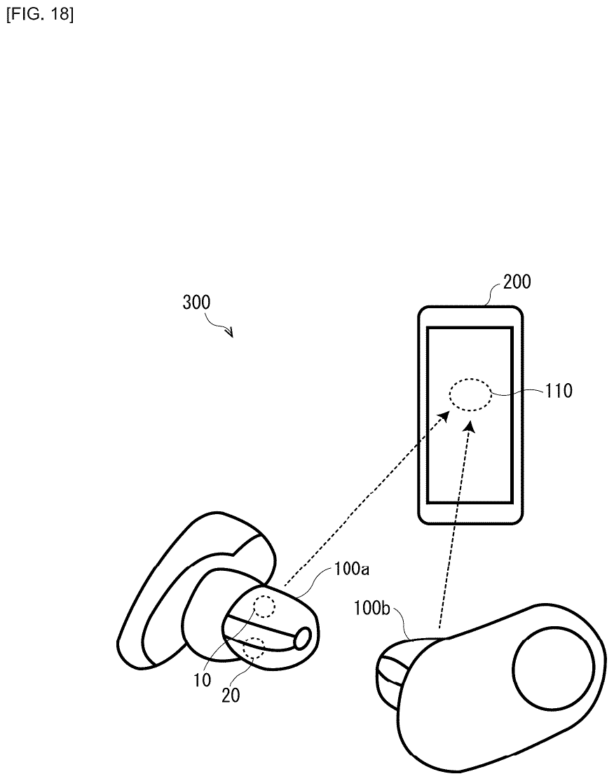

The biological signal detection device 1 may be mounted on a small-sized earphone device, such as, for example, a TWS (True Wireless Stereo), as in an example illustrated in FIG. 17. As in the example illustrated in FIG. 17 or FIG. 18, the biological signal detection device 1 may be configured as a biological signal detection system 300 including: the measurer 100a; the measurer 100b; the electronic apparatus 200 including the signal processor 110; and the like. The biological signal detection system 300 may include, for example, the estimator 120, the display unit, the sound output unit, and the like that have been described above.

Although the description has been given hereinabove of the present disclosure with reference to the embodiment and modification example, the present technology is not limited to the foregoing embodiment and the like, and may be modified in a wide variety of ways. For example, although the above-described modification examples have been described as the modification examples of the above-described embodiment, the configurations of the respective modification examples may be combined as appropriate. In addition, the present disclosure has applicability not only to the human body, but also to a living body other than the human body, for example, animals such as pets and domestic animals.

The biological signal detection device according to an embodiment of the present disclosure includes a first measurer and a second measurer. The first measurer and the second measurer each include a first electrode and a second electrode that are configured to be in contact with a living body, and a first passive element that is electrically coupled between the second electrode and a reference potential line. The first measurer and the second measurer are each configured to output a first signal based on an electric potential of the first electrode. Accordingly, it is possible to achieve the biological signal detection device having favorable detection performance.

The biological signal detection system according to an embodiment of the present disclosure includes a first measurer, a second measurer, and a signal processing device. The first measurer and the second measurer each include a first electrode and a second electrode that are configured to be in contact with a living body, and a first passive element that is electrically coupled between the second electrode and a reference potential line. The first measurer and the second measurer are each configured to output a first signal based on an electric potential of the first electrode. The signal processing device is configured to generate a second signal related to the living body, on a basis of the first signal to be generated by the first measurer and the first signal to be generated by the second measurer. Accordingly, it is possible to achieve the biological signal detection system having favorable detection performance.

It is to be noted that the effects described in the present specification are merely examples and are not limited to the description, and other effects may be obtained. Further, the present disclosure may have the following configuration.

(1)

A biological signal detection device including:
a first measurer; and
a second measurer,
the first measurer and the second measurer each including
   a first electrode and a second electrode that are configured to be in contact with a living body, and
   a first passive element that is electrically coupled between the second electrode and a reference potential line, in which
the first measurer and the second measurer are each configured to output a first signal based on an electric potential of the first electrode.
   (2)

The biological signal detection device according to (1), further including a signal processor configured to generate a second signal related to the living body, on a basis of the first signal to be generated by the first measurer and the first signal to be generated by the second measurer.

(3)

The biological signal detection device according to (1) or (2), in which the signal processor is configured to generate the second signal based on a difference between the first signal to be generated by the first measurer and the first signal to be generated by the second measurer.

(4)

The biological signal detection device according to any one of (1) to (3), in which
the first measurer includes a supplier that is electrically coupled to the reference potential line and is configured to supply a voltage, and
the first passive element is electrically coupled between the second electrode and the supplier.
   (5)

The biological signal detection device according to any one of (1) to (4), in which the first passive element includes a resistor or a capacitor.

(6)

The biological signal detection device according to any one of (1) to (5), in which
the first measurer includes
   a third electrode configured to be in contact with the living body, and
   a second passive element that is electrically coupled between the third electrode and the reference potential line.
      (7)

The biological signal detection device according to (6), in which the second passive element includes a resistor or a capacitor.

(8)

The biological signal detection device according to (6) or (7), in which
the first measurer includes a supplier that is electrically coupled to the reference potential line and is configured to supply a voltage,
the first passive element is electrically coupled between the second electrode and the supplier, and
the second passive element is electrically coupled between the third electrode and the supplier.
   (9)

The biological signal detection device according to any one of (1) to (8), in which the first measurer includes a first amplifier circuit configured to output a third signal based on the electric potential of the first electrode and an electric potential of the second electrode.

(10)

The biological signal detection device according to any one of (1) to (9), in which
the first measurer includes an AD converter circuit configured to convert the third signal into a digital signal, and
the first measurer is configured to output, as the first signal, the third signal that has been converted into the digital signal.
   (11)

The biological signal detection device according to any one of (1) to (10), in which
the first measurer includes
   a first amplifier circuit configured to output a third signal based on the electric potential of the first electrode and an electric potential of the second electrode, and
   a second amplifier circuit configured to output a fourth signal based on the third signal and the electric potential of the first electrode.
      (12)

The biological signal detection device according to (11), in which
the first measurer includes an AD converter circuit configured to convert the fourth signal into a digital signal, and
the first measurer is configured to output, as the first signal, the fourth signal that has been converted into the digital signal.
   (13)

The biological signal detection device according to any one of (1) to (12), in which
the first measurer includes
   a third electrode and a fourth electrode that are configured to be in contact with the living body,
   a first amplifier circuit configured to output a third signal based on an electric potential of the third electrode and an electric potential of the fourth electrode, and
   a second amplifier circuit configured to output a fourth signal based on the third signal and the electric potential of the first electrode.
      (14)

The biological signal detection device according to (13), in which
the first measurer includes an AD converter circuit configured to convert the fourth signal into a digital signal, and
the first measurer is configured to output, as the first signal, the fourth signal that has been converted into the digital signal.
   (15)

The biological signal detection device according to any one of (1) to (14), in which an impedance of the first passive element is greater than an impedance between the living body and the second electrode.

(16)

The biological signal detection device according to any one of (1) to (15), in which an impedance of the first passive element is greater than or equal to ten times an impedance between the living body and the second electrode.

(17)

A biological signal detection system including:
a first measurer and a second measurer each including a first electrode and a second electrode that are configured to be in contact with a living body, and a first passive element that is electrically coupled between the second electrode and a reference potential line, the first measurer and the second measurer each being configured to output a first signal based on an electric potential of the first electrode; and
a signal processing device configured to generate a second signal related to the living body, on a basis of the first signal to be generated by the first measurer and the first signal to be generated by the second measurer.
   (18)

The biological signal detection system according to (17), in which the signal processing device is configured to generate the second signal based on a difference between the first signal to be generated by the first measurer and the first signal to be generated by the second measurer.

(19)

The biological signal detection system according to (17) or (18), in which
the first measurer includes a supplier that is electrically coupled to the reference potential line and is configured to supply a voltage, and
the first passive element is electrically coupled between the second electrode and the supplier.
   (20)

The biological signal detection system according to any one of (17) to (19), in which
the first measurer includes
   a third electrode that is configured to be in contact with the living body, and
   a second passive element that is electrically coupled between the third electrode and the reference potential line.
      (21)

The biological signal detection system according to (20), in which
the first measurer includes a supplier that is electrically coupled to the reference potential line and is configured to supply a voltage,
the first passive element is electrically coupled between the second electrode and the supplier, and
the second passive element is electrically coupled between the third electrode and the supplier.

This application claims the benefit of Japanese Priority Patent Application JP2023-075322 filed with the Japan Patent Office on Apr. 28, 2023, the entire contents of which are incorporated herein by reference.

It should be understood by those skilled in the art that various modifications, combinations, sub-combinations, and alterations may occur depending on design requirements and other factors insofar as they are within the scope of the appended claims or the equivalents thereof.

1. A biological signal detection device comprising:
a first measurer; and
a second measurer,
the first measurer and the second measurer each including
a first electrode and a second electrode that are configured to be in contact with a living body, and
a first passive element that is electrically coupled between the second electrode and a reference potential line, wherein
the first measurer and the second measurer are each configured to output a first signal based on an electric potential of the first electrode.

2. The biological signal detection device according to claim 1, further comprising a signal processor configured to generate a second signal related to the living body, on a basis of the first signal to be generated by the first measurer and the first signal to be generated by the second measurer.

3. The biological signal detection device according to claim 2, wherein the signal processor is configured to generate the second signal based on a difference between the first signal to be generated by the first measurer and the first signal to be generated by the second measurer.

4. The biological signal detection device according to claim 1, wherein
the first measurer includes a supplier that is electrically coupled to the reference potential line and is configured to supply a voltage, and
the first passive element is electrically coupled between the second electrode and the supplier.

5. The biological signal detection device according to claim 1, wherein the first passive element comprises a resistor or a capacitor.

6. The biological signal detection device according to claim 1, wherein
the first measurer includes
a third electrode configured to be in contact with the living body, and
a second passive element that is electrically coupled between the third electrode and the reference potential line.

7. The biological signal detection device according to claim 6, wherein the second passive element comprises a resistor or a capacitor.

8. The biological signal detection device according to claim 6, wherein
the first measurer includes a supplier that is electrically coupled to the reference potential line and is configured to supply a voltage,
the first passive element is electrically coupled between the second electrode and the supplier, and
the second passive element is electrically coupled between the third electrode and the supplier.

9. The biological signal detection device according to claim 1, wherein the first measurer includes a first amplifier circuit configured to output a third signal based on the electric potential of the first electrode and an electric potential of the second electrode.

10. The biological signal detection device according to claim 9, wherein
the first measurer includes an AD converter circuit configured to convert the third signal into a digital signal, and
the first measurer is configured to output, as the first signal, the third signal that has been converted into the digital signal.

11. The biological signal detection device according to claim 1, wherein
the first measurer includes
a first amplifier circuit configured to output a third signal based on the electric potential of the first electrode and an electric potential of the second electrode, and
a second amplifier circuit configured to output a fourth signal based on the third signal and the electric potential of the first electrode.

12. The biological signal detection device according to claim 11, wherein
the first measurer includes an AD converter circuit configured to convert the fourth signal into a digital signal, and
the first measurer is configured to output, as the first signal, the fourth signal that has been converted into the digital signal.

13. The biological signal detection device according to claim 1, wherein
the first measurer includes
a third electrode and a fourth electrode that are configured to be in contact with the living body,
a first amplifier circuit configured to output a third signal based on an electric potential of the third electrode and an electric potential of the fourth electrode, and
a second amplifier circuit configured to output a fourth signal based on the third signal and the electric potential of the first electrode.

14. The biological signal detection device according to claim 13, wherein
the first measurer includes an AD converter circuit configured to convert the fourth signal into a digital signal, and
the first measurer is configured to output, as the first signal, the fourth signal that has been converted into the digital signal.

15. The biological signal detection device according to claim 1, wherein an impedance of the first passive element is greater than an impedance between the living body and the second electrode.

16. The biological signal detection device according to claim 1, wherein an impedance of the first passive element is greater than or equal to ten times an impedance between the living body and the second electrode.

17. A biological signal detection system comprising:
a first measurer and a second measurer each including a first electrode and a second electrode that are configured to be in contact with a living body, and a first passive element that is electrically coupled between the second electrode and a reference potential line, the first measurer and the second measurer each being configured to output a first signal based on an electric potential of the first electrode; and
a signal processing device configured to generate a second signal related to the living body, on a basis of the first signal to be generated by the first measurer and the first signal to be generated by the second measurer.

18. The biological signal detection system according to claim 17, wherein the signal processing device is configured to generate the second signal based on a difference between the first signal to be generated by the first measurer and the first signal to be generated by the second measurer.

19. The biological signal detection system according to claim 17, wherein
the first measurer includes a supplier that is electrically coupled to the reference potential line and is configured to supply a voltage, and
the first passive element is electrically coupled between the second electrode and the supplier.

20. The biological signal detection system according to claim 17, wherein
the first measurer includes
a third electrode that is configured to be in contact with the living body, and
a second passive element that is electrically coupled between the third electrode and the reference potential line.
